(19) United States
(12) Patent Application Publication
(10) Pub. No.: US 2016/0166750 A1
(43) Pub. Date: Jun. 16, 2016

(54) Oxygenator

(71) Applicant: UNIVERSITEIT GENT, Gent (BE)

(72) Inventor: Filip DE SOMER, Gent (BE)

(21) Appl. No.: 14/905,709

(22) PCT Filed: Jul. 15, 2014
(86) PCT No.: PCT/EP2014/065117

§ 371 Date: Jan. 15, 2016

(30) Foreign Application Priority Data
Jul. 18, 2013 (EP) 13176971.3

(51) Int. Cl. A61M 1/16; A61M 1/36

(52) U.S. Cl. CPC A61M 1/1629; A61M 1/1698; A61M 1/1601; A61M 1/3635; A61M 2205/3372

(57) ABSTRACT

A device is described for oxygenating and controlling a temperature of blood in an extracorporeal blood circuit. The device comprises a filter element provided for filtering gaseous microemboli and solid particles from the blood. The filter element is disposed downstream of the distributor and upstream of the heat exchanger.

### FIELD OF THE INVENTION

The invention relates to extracorporeal blood circuits. More specifically it relates to devices for oxygenating and filtering blood in an extracorporeal blood circuit.

### BACKGROUND OF THE INVENTION

An extracorporeal blood circuit is commonly used during cardiopulmonary bypass to withdraw blood from the venous portion of the patient and return the blood to the arterial portion.

Blood oxygenators are disposable components of extracorporeal blood circuits and are used to oxygenate blood. An oxygenator comprises a number of gas exchange elements, for instance a number of microporous hollow fibers, around which blood flow is directed and through which an oxygen-rich gas mixture is passed. Carbon dioxide in the blood arriving from the patient in the blood oxygenator diffuses across the gas exchange elements into the stream of oxygen-rich gas. At the same time oxygen transfers from an oxygen-rich gas mixture across the gas exchange elements into the blood.

Prior to interfacing with the oxygenator, the patient's blood is usually continuously pumped through a heat exchanger. The function of the heat exchanger is to control the temperature of the blood in a desired direction. The heat exchanger comprises a number of heat exchange elements, for instance a number of plastic or metal tubes through which a suitable heat transfer fluid is pumped, separate from but in heat exchange relationship with the blood flow which is directed adjacent the heat exchange elements. In this way, heat exchange is established and the blood is brought to the preferred temperature.

Conventionally, the extracorporeal blood circuit further comprises a filter device provided to remove gross air, solid particles (for instance in the order 20-40 micrometer), as well as to trap and remove gaseous microemboli. The filter device is usually a physically separated component from the oxygenator and heat exchanger component. In order to minimize the extracorporeal blood circuit's prime volume, devices have been proposed that integrate the filter device with the oxygenator and/or the heat exchanger component.

US 2012/0193289A1 describes such an integrated device for use in an extracorporeal blood circuit comprising an oxygenator and heat exchanger component integrated in the same housing. The gas exchange elements of the oxygenator are concentrically arranged around the heat exchanger component. Part of the housing is shaped to form a de-airing region to remove gross air from the blood flow prior to interaction with the heat exchanger and oxygenator. Gross air removal is understood by those skilled in the art to include relatively large volume air bubbles that would otherwise simulate a CVR draining. The apparatus described in US 2012/0193289 may further comprise a filter component, akin to conventional filter components, to trap gaseous microemboli and small particles. The filter component can be located around the gas exchange elements of the oxygenator, between the heat exchanger and the oxygenator or between layers of the gas exchange elements of the oxygenator.

### SUMMARY OF THE INVENTION

It is an object of embodiments of the present invention to provide an integrated oxygenating/heat exchanging device in which gaseous microemboli and/or small particles can be removed in a more efficient way.

The above objective is accomplished by a device according to the present invention.

The present invention relates to a device for controlling a temperature of blood in an extracorporeal blood circuit. The device comprises a housing, a blood inlet, a distributor, a filter element, a heat exchanger and an oxygenator. The blood inlet is disposed within the housing and provided for receiving venous blood. The distributor is disposed within the housing and arranged downstream of the blood inlet and is in fluid communication therewith. The distributor is provided for distributing blood towards the heat exchanger. The heat exchanger is disposed within the housing and arranged downstream of the distributor. The heat exchanger comprises a plurality of heat exchange elements for controlling the temperature of the blood. In some embodiments, devices comprise an oxygenator being disposed within the housing and arranged downstream of the heat exchanger. The device thus may be suitable for oxygenating blood in an extracorporeal blood circuit. The oxygenator may comprise at least one gas exchange element for oxygenating the blood. In some alternative embodiments, as will be described in the second aspect, the device does not comprise an oxygenator but may be connectable to an oxygenator. The filter element is disposed within the housing and provided for filtering gaseous microemboli and solid particles from the blood. The filter element is disposed downstream of the distributor and upstream of the heat exchanger. It is an advantage of embodiments of the present invention that air bubbles and gaseous emboli become less fractionated, facilitating their removal compared to prior art devices. It is an advantage of embodiments of the present invention that the efficiency of a bubble trap or de-airing region, if any, can be improved. It is an advantage of embodiments of the present invention that less constraints to the type of filter element used have to be put compared to prior art devices. It is advantage of embodiments of the present invention that the filter element can be adapted to remove additional particles compared to prior art devices. It is an advantage of embodiments of the present invention that the filter element results in less blood damage compared to prior art devices.

The filter element of the device may cover at least part of the outer surface of the distributor. The filter element of the device may cover at least part of the inner surface of the heat exchanger. It is an advantage of embodiments of the present invention that, by positioning the filter element in close contact with the distributor and/or heat exchanger, additional support to the filter element may be realized. It is an advantage of embodiments of the present invention that the filter element may be made thinner compared to prior art filter elements used, resulting in less blood damage when passing the filter element.

The filter element may be a filter with a mean pore size in the range 20 to 150 μm.

The filter element may be a thin filter element supported by the heat exchanger.

The filter element may be a filter element having a thickness between 35 μm and 15 μm, e.g. between 30 μm and 15 μm.

The filter element and the heat exchanger may comprise or may be built from fibrous elements.

In a second aspect, the present invention also relates to a heat exchange device. The heat exchange device is suitable for controlling a temperature of blood in an extracorporeal blood circuit. The heat exchange device according to a second aspect of the present invention comprises a housing and a distributor, a filter element and a heat exchanger, disposed within the housing. The distributor may be arranged downstream of a blood inlet, the blood inlet being part of the housing or not, and in fluid communication therewith. The distributor is provided for distributing blood towards the heat exchanger. The heat exchanger is disposed within the housing and arranged downstream of the distributor. The heat exchanger comprises a plurality of heat exchange elements for controlling the temperature of the blood. The filter element is disposed within the housing and provided for filtering gaseous microemboli and solid particles from the blood. The filter element is disposed downstream of the distributor and upstream of the heat exchanger. It is an advantage of embodiments of the present invention that air bubbles and gaseous emboli become less fractionated, facilitating their removal compared to prior art devices. It is an advantage of embodiments of the present invention that the efficiency of a bubble trap or de-airing region, if any, can be improved. It is an advantage of embodiments of the present invention that less constraints to the type of filter element used have to be put compared to prior art devices. It is advantage of embodiments of the present invention that the filter element can be adapted to remove additional particles compared to prior art devices. It is an advantage of embodiments of the present invention that the filter element results in less blood damage compared to prior art devices.

The heat exchange device according to a second aspect of the present invention may be part of a device according to a first aspect of the present invention. The heat exchange device according to a second aspect of the present invention may be suitable for use with an oxygenator. The oxygenator and the heat exchange device may be integrated in the same housing or may be two separate components suitably connected. The oxygenator may be arranged downstream of the heat exchange device and/or the heat exchanger. The oxygenator comprises at least one gas exchange element for oxygenating the blood.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

FIG. 1 shows a schematic drawing of a first embodiment of a device according to the present invention.

FIG. 2 shows a schematic drawing of a second embodiment of a device according to the present invention.

The drawings are only schematic and are non-limiting. In the drawings, the size of some of the elements may be exaggerated and not drawn on scale for illustrative purposes.

Any reference signs in the claims shall not be construed as limiting the scope.

In the different drawings, the same reference signs refer to the same or analogous elements.

### DETAILED DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Moreover, the terms top, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Where in the present invention reference is made to a location downstream of a component of a device, it is meant a location of the component away from the inlet with respect to the flow. Where in the present invention reference is made to a location upstream of a component of a device, it is meant a location towards the inlet of the device with respect to the flow.

By way of illustration standard and optional features will further be described with reference to the drawings, embodiments of the present invention not being limited thereto.

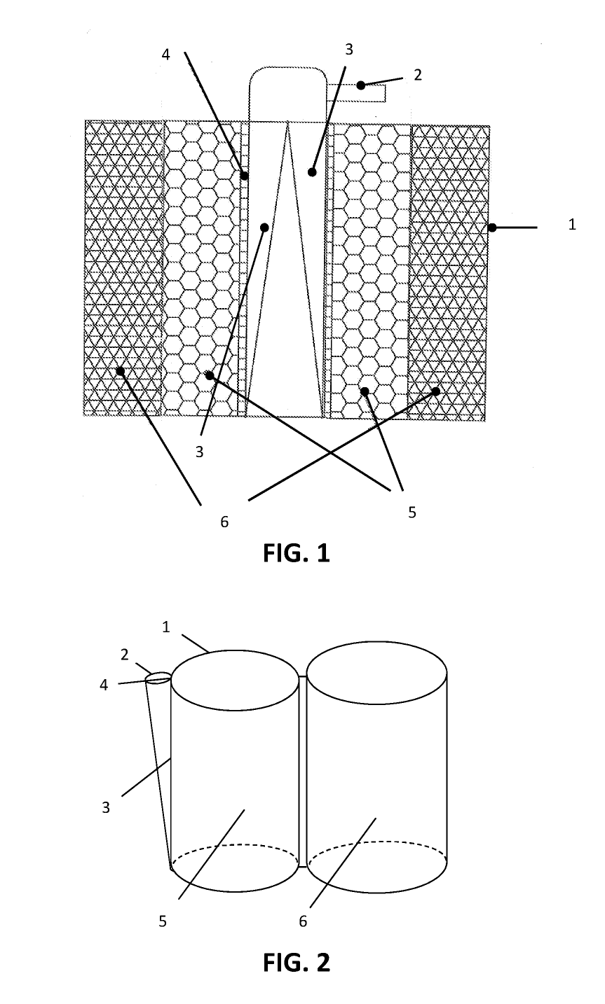

Referring to FIG. 1, a schematic drawing of an exemplary device according to a first embodiment of the present invention is shown.

Referring to FIG. 2, a schematic drawing of an exemplary device according to a second embodiment of the present invention is shown.

The device according to an embodiment of the present invention comprises a housing 1, a blood inlet 2, a distributor 3, a filter element 4, a heat exchanger 5 and, according to some embodiment, an oxygenator 6. Alternatively, the device may be connectable to an oxygenator, as will be described in the second aspect. The device may further comprise a blood outlet. A blood flow path is defined from the blood inlet to the blood outlet, over consecutively the distributor, the filter element, the heat exchanger and the oxygenator.

The device according to an embodiment of the present invention comprises a blood inlet 2 for receiving blood from a venous portion of an extracorporeal blood circuit, i.e. for receiving venous blood. The blood inlet 2 may be arranged for directing incoming blood in a certain direction towards the distributor 3 and the filter element 4.

The device according to an embodiment of the present invention further comprises a distributor 3 for distributing blood coming from the blood inlet 2 towards the one or more heat exchange elements of the heat exchanger 5. The distributor is in fluid contact with the blood inlet 2. Distributing the blood may advantageously be done in such a way that the blood velocity is substantially equal at different points of the heat exchanger. The distributor 3 as shown in FIG. 1 is for instance formed by a cylindrical gap between the outer surface of the inner core of the device and the inner surface of the cylindrical heat exchanger. The width of the gap decreases from top to bottom, as a result of which the blood velocity at different heights of the heat exchanger is substantially equal. The distributor 3 as shown in FIG. 2 is for instance formed by a conical space decreasing from top to bottom, as a result of which the blood velocity at different heights of the heat exchanger is substantially equal. Alternatively, or in addition thereto, the distributor may comprise a manifold for further controlling the velocity profile or realizing an alternative velocity profile. Nevertheless, if another velocity profile would be preferred, the latter can also be established and does not limit embodiments of the present invention.

In a device according to an embodiment of the present invention the distributor may take whatever shape as considered suitable by the person skilled in the art. The distributor may for instance be formed by a distribution manifold comprising one or more orifices that can be closed more or less depending on the desired blood velocity.

The device according to an embodiment of the present invention further comprises a heat exchanger 5. The heat exchanger 5 is arranged downstream of the distributor 3. The heat exchanger 5 may comprise a number of heat exchange elements through which a heat exchange fluid is passed, such as for instance water. Alternatively to a heat exchange fluid, other heat exchange means may be used such as for instance thermo energy which is supplied to the heat exchange elements. The purpose of the heat exchange elements is to control the temperature of the blood passing over and/or between the heat exchange elements. This is done by transferring heat from or to the heat exchange fluid running through the heat exchange elements/heat exchange means to or from the blood flowing over and/or between the heat exchange elements.

The heat exchange elements may take the form of fibers, tubes, capillaries, compartments, but are not limited thereto. The heat exchange elements may be made of a heat conducting material, such as a heat conducting metal or polymer.

The heat exchange elements of the exemplary devices shown in FIG. 1 and FIG. 2 may for instance be concentrically arranged around an inner core. The heat exchange elements shown in FIG. 1 and FIG. 2 may comprise a plurality of hollow channels through which heat exchange fluid is passed. The channels may be arranged in such a way that blood coming from the distributor, can flow over and between the channels. The heat exchange elements may be arranged such that blood coming from the distributor is directed in a substantially radial direction of the heat exchanger as for instance may be the case in FIG. 1, or in a substantially perpendicular direction to the flow of the heat exchanger fluid and/or tangential direction of the heat exchanger as for instance may be the case in FIG. 2. The heat exchange elements may be wound on an inner core of the device or may be pre-arranged and assembled over an inner core.

The device according to embodiments of the present invention further comprises a filter element 4 provided for removing gaseous micro-emboli and solid particles. The filter element 4 is arranged between the distributor 3 and the heat exchanger 5. The filter element 4 may be arranged downstream of the distributor 3 and upstream of the heat exchanger 5. The filter element 4 may have any shape considered suitable for the person skilled in the art, such as flat, cylindrical or conical, and the shape used may depend on the shape of the other components in the device. The filter element 4 shown in FIG. 1 for instance has a cylindrical shape. The filter element 4 may be arranged in the gap between the outer surface of the inner core of the device and the inner surface of the heat exchanger as shown in FIG. 1. The filter element 4 may cover part or the whole surface of the distributor 3. The filter element 4 as shown in FIG. 2 may fit the inner surface of the conical gap forming the distributor 3 or only part thereof and or cover a transition contact area between distributor and heat exchanger. The filter element 4 may cover at least part of or the whole inner surface of the heat exchanger 5. The filter element 4 may cover at least part of or the whole outer surface of the distributor 3. The filter element 4 is arranged such that blood passing the filter element has not yet passed the heat exchanger 5. Positioning the filter element 4 between the distributor 3 and the heat exchanger 5 has the advantage that air bubbles and gaseous emboli are less fractionated, facilitating their removal compared to prior art devices. Positioning the filter element between the distributor and the heat exchanger has the advantage that the distribution of the blood through the heat exchanger as well as through the oxygenator is improved. Positioning the filter element between the distributor and the heat exchanger has the advantage that the efficiency of a bubble trap or de-airing region, if any, can be improved.

Positioning the filter element prior to the heat exchanger and the oxygenator may result in a more efficient removal of gaseous micro-emboli compared to prior art devices.

Positioning the filter element prior to the heat exchanger and the oxygenator may put less constraints to the type of filter element used, for instance filter elements with larger or smaller pore sizes may be used, resulting in the same or even better efficiency compared to prior art devices and/or removal of additional particles, such as for instance solid particles, compared to prior art devices and/or less blood damage when passing the filter element compared to prior art devices.

The filter element 4 may have properties, such as but not limited to material properties, mean pore size, basis weight, comparable to conventional arterial filters used in extracorporeal blood treatment systems. The filter element 4 may be made of any material considered suitable by the person skilled in the art, for instance a conventional arterial filter filtration material, such as for instance a hemocompatible polymer material. The filter element may have a mean pore size in the range of 20-150 μm. The filter element may have a mean pore size in the range of 20-40 μm, comparable to prior art device, or a larger pore size in the range of 40-150 μm.

In a particular embodiment, the filter element is arranged as a fibrous filter element followed by a fibrous heat exchanger. The latter can be efficiently manufactured. Positioning the fibrous filter in close contact with a heat exchanger may have the advantage that the heat exchanger provides a support for the filter element. As a result of the additional support by the heat exchanger, the filter element may be made thinner compared to prior art devices. The thickness of the filter element may for instance be in the range between 15 μm-35 μm; between 15 μm-30 μm, or between 20 μm and 30 μm. Such arrangement may result in less blood damage when passing the filter element.

The device according to some embodiments of the present invention further comprises an oxygenator 6. The oxygenator is arranged downstream of the heat exchanger. The oxygenator 6 comprises a number of gas exchange elements through which an oxygen-rich gas is passed. The gas exchange elements shown in FIG. 1 are concentrically arranged around the cylindrical heat exchanger and have a cylindrical shape. The gas exchange elements shown in FIG. 1 may for instance comprise a plurality of hollow fibers which are wound directly onto the heat exchanger. Blood coming from the heat exchanger is passed over the gas exchange elements. The gas exchange elements are made of semi-permeable membranes which are able to transfer oxygen from the oxygen-rich gas to the blood flowing over the gas exchange elements and carbon dioxide from the blood to the oxygen-rich gas. The gas exchange elements shown in FIG. 2 may for instance be concentrically arranged around an inner core, different from the inner core of the heat exchange elements. The gas exchange elements shown in FIG. 2 may for instance comprise a plurality of hollow fibers which are wound directly onto an inner core. The gas exchange elements may be wound on an inner core of the device or may be pre-arranged and assembled over the inner core.

The device according to an embodiment of the present invention may further comprise a blood outlet for receiving blood coming from the oxygenator and returning it to an arterial portion of the extracorporeal blood circuit.

FIG. 1 and FIG. 2 show an integrated oxygenator/heat exchanger device, i.e. a device in which the oxygenator and the heat exchanger are not physically separated components of the extracorporeal blood circuit but are integrated in one and the same device or housing.

In a second aspect of the invention the invention relates to a heat exchange device comprising a housing, a distributor, a filter element and a heat exchanger, disposed within the housing for controlling a temperature of blood in an extracorporeal blood circuit. The housing, the distributor, the filter element and the heat exchanger of the heat exchange device according to a second aspect of the invention may have similar properties and advantages as the corresponding components of the device according to a first aspect of the present invention. The heat exchange device may be suitable for use with an oxygenator and/or may be integrated in the same housing with the oxygenator. Alternatively, the heat exchange device and the oxygenator may be two separate components suitably connected as such forming a device for both oxygenating and controlling a temperature of blood in an extracorporeal blood circuit.

In one aspect, the present invention also relates to a device according to the first or second aspect for controlling a temperature of blood in an extracorporeal blood circuit and for oxygenating and controlling a temperature of blood in an extracorporeal blood circuit.

1. A device for controlling a temperature of blood flow in an extracorporeal blood circuit, the device comprising:
a housing (1)
an inlet (2) disposed within the housing for receiving venous blood
a distributor (3) disposed within the housing (1) and arranged downstream of the inlet (2), the distributor (3) being fluidly connected to the inlet and provided for distributing the blood toward the heat exchanger (5)
a heat exchanger (5) disposed within the housing (1) and arranged downstream of the distributor (3) , the heat exchanger (5) comprising a plurality of heat exchange elements for controlling the temperature of the blood,
the device being connectable to an oxygenator comprising at least one gas exchange element for oxygenating the blood,
the device further comprising a filter element (4) disposed within the housing (1) and provided for filtering gaseous microemboli and solid particles from the blood characterized in that
the filter element (4) is disposed downstream of the distributor (3) and upstream of the heat exchanger (5).

2. A device according to claim 1, wherein the filter element covers at least part of the outer surface of the distributor.

3. A device according to any of claims 1 to 2, wherein the filter element covers at least part of the inner surface of the heat exchanger.

4. A device according to any of claims 1 to 3, wherein the filter element covers at least part of a transition contact area between the distributor and the heat exchanger.

5. A device according to any of claims 1 to 4, wherein the device furthermore comprises the oxygenator comprising the at least one gas exchange element for oxygenating the blood.

6. A device according to any of claims 5, wherein the oxygenator is disposed within the housing and arranged downstream the heat exchanger (5).

7. A device according to any of claims 1 to 6, wherein the filter element is a filter with a mean pore size in the range 20 to 150 μm.

8. A device according to any of claims 1 to 7, wherein the filter element is a thin filter element supported by the heat exchanger.

9. A device according to claim 8, wherein the filter element is a filter element having a thickness between 35 μm and 15 μm, e.g. between 30 μm and 15 μm.

10. A device according to any of the previous claims, wherein the filter element and the heat exchanger are fibrous elements.

11. Use of a device according to any of claims 1 to 6 for controlling a temperature of blood in an extracorporeal blood circuit.

12. Use of a device according to any of claims 5 to 6 for oxygenating and controlling a temperature of blood in an extracorporeal blood circuit.

13. An artificial lung comprising a device according to any of claims 1 to 10.
